(19) United States
(12) Patent Application Publication
(10) Pub. No.: US 2009/0221882 A1
(43) Pub. Date: Sep. 3, 2009

(54) Implantable Biosensor Assembly and Health Monitoring system and Method including same

(76) Inventor: Dan Gur Furman, Gedera (IL)

Correspondence Address: BAKER & DANIELS LLP, 300 NORTH MERIDIAN STREET, SUITE 2700, INDIANAPOLIS, IN, 46204, US

(21) Appl. No.: 12/096,474

(22) PCT Filed: Dec. 10, 2006
(86) PCT No.: PCT/IL2006/001416

§ 371 Date: Jan. 5, 2009

Related U.S. Application Data
(60) Provisional application No. 60/748,218, filed on Dec. 8, 2005.

(51) Int. Cl. A61B 5/00

(52) U.S. Cl. 600/301

(57) ABSTRACT

A biosensor assembly implantable in a subject's body for monitoring a medical condition of the subject includes a housing, one or more biosensors, a reference-data storage device for storing reference data corresponding to a normal condition of the subject, a transmitter, a battery, and a processor designed: (a) to compare the measured data, as it is sensed by the biosensors, with the reference data stored in said reference-data storage device; (b) to determine whether the measured data indicates a normal medical condition or an abnormal medical condition in the subject: and (c) upon determining that the measured data indicates an abnormal condition, to actuate the transmitter to transmit an alarm signal to a central station, to the subject, and/or to a caregiver

### FIELD AND BACKGROUND OF THE INVENTION

The present invention relates to implantable biosensor assemblies, and also to a health monitoring system and method including such assemblies.

For medical reasons in vivo parameters of a subject may need to be monitored over a period of time; for example, such monitoring may be necessary in a subject who has occasional atypical cardiac beats. By providing continuous monitoring, medical personnel determine if there is a tendency for production of sustained atypical beats in a life-endangering fashion. Medical personnel also use the monitoring results to establish a proper course of treatment.

In measuring ECG over a period of time using an ECG sensor, the subject is usually fitted with a halter that continuously measures and stores the heart electrical activity, for example over a 24 hour period. Upon completion of the measurement period, the halter is returned to a care providing organization where the 24-hour activity is analyzed, and a plan of action is formulated.

A prime drawback of such a system is that the subject must actively take part in ensuring that the halter is worn properly. Also, the sensors must remain in position; the halter battery must be functioning; and the halter must be physically returned to a caregiver. Should the subject experience a cardio-vascular accident during the monitoring, the subject may be unable to contact the caregiver for help in sufficient time.

Based upon the above scenario, particularly when there is a chance for untoward, life-threatening events to occur, there is a need to provide ongoing monitoring in a manner such that the monitoring occurs without the subject's active input, automatically and autonomously.

One prior art device that measures heart rate is the “Reveal” monitor by Medtronic (Minneapolis, Minn., USA). This device comprises an implantable heart monitor used, for example, in determining if fainting in a subject is related to a heart rhythm problem. The Reveal monitor continuously monitors the rate and rhythm of the heart for up to 14 months. After waking from a fainting episode, the subject places a first recorder device external to. the skin over the implanted Reveal monitor and presses a button to transfer data from the monitor to the recorder. The subject. gives the first recorder to a physician who provides the subject with a second (empty) recorder. The physician then analyzes the information stored on the first recorder to determine whether abnormal heart rhythm has been recorded. There are several drawbacks in the Reveal system:

1. It is important for the subject to keep the recorder handy at all times (clipped to the clothes or looped over a belt). If the subject fails to do so, data can be irrevocably lost.

2. The use of the recorder is neither automatic nor autonomic, and therefore requires the subject to be conscious. In cases of fainting, no data can be reliably collected until the subject is transferred to a hospital, the subject physician is contacted, and a recorder is provided. Unfortunately, the anticipated delays could negatively impact the subject, possibly leading to death.

Another known type of implantable biosensor monitoring device is a transponder-type device, in which a transponder is implanted in a patient and subsequently accessed with a hand-held electromagnetic reader in a non-invasive manner. An example of the latter type of device is described in U.S. Pat. No. 5,833,603. However, this type of device does not allow automatic and autonomic measurement of the subject's parameters, nor automatic and autonomic transfer and analysis of the parameters by medical personnel, nor automatic and autonomic treatment in the event an abnormal condition is detected, e.g., an electrical shock treatment in case of cardiac arrest.

### OBJECTS AND BRIEF SUMMARY OF THE PRESENT INVENTION

An object of the present invention is to provide an implantable biosensor assembly having advantages in one or more of the above respects. Another object of the invention is to provide a health monitoring system including such an implantable biosensor, and a further object is to provide an improved method of health monitoring a plurality of subjects by the use of implantable biosensors.

According to one aspect of the present invention, there is provided a biosensor assembly implantable in a subject's body for monitoring a medical condition of the subject, comprising: a housing sized and configured for implantation in the subject's body; the housing including: at least one biosensor for sensing and measuring a medical parameter related to the monitored medical condition, and for producing an output corresponding to the measured parameter; a measured-data storage device for receiving the output of the biosensor and for storing measured data therein corresponding to the measured parameter; a reference-data storage data device for storing reference data corresponding to a normal medical condition of the respective subject; a transmitter including an antenna for transmitting data externally of the subject's body; a battery for powering the transmitter; and a processor designed: (a) to compare the measured data, as it is sensed by the biosensor, with the reference data stored in the reference-data storage device; (b) to determine whether the measured data indicates a normal medical condition or an abnormal medical condition in the subject; and (c) upon determining that the measured data indicates an abnormal medical condition, to actuate the transmitter to transmit a signal externally of the subject's body indicating that an abnormal medical condition has been determined to be present.

According to further features in the preferred embodiment of the invention described below, the housing is sized and configured for implantation subcutaneously in the subject's body, and includes a plurality of biosensors for sensing and measuring a plurality of different medical parameters all related to the medical condition of the subject being monitored; and the processor is designed to utilize the outputs of the plurality of biosensors in determining whether the measured data indicates a normal medical condition or an abnormal medical condition in the subject.

In the described preferred embodiments, the plurality of biosensors include: an ECG sensor for sensing and measuring the ECG signals of the subject, an oxygen-saturation sensor for sensing and measuring oxygen saturation of the subject's blood, a Doppler sensor for sensing and measuring blood flow velocity in the subject, and a temperature sensor for sensing and measuring the temperature of the subject. The processor may process the outputs of a plurality of the biosensors for sensing and measuring the heart rate and/or the blood pressure of the subject in accordance with known algorithms utilizing the above parameters. It will be appreciated that other biosensors could be used to measure other parameters, and other medical conditions could be monitored.

In one described preferred embodiment, the transmitter is a cellular modem for transmitting the data via the cellular telephone network; and the implantable biosensor includes a cellular modem receiver for receiving calls from the cellular telephone network. In another described embodiment, the transmitter is a GPS (ground positioning system) transmitter for transmitting the data via the GPS satellite system; and the GPS transmitter also transmits signals via the GPS satellite system identifying the respective biosensor assembly and its current location.

According to a still further feature in the described preferred embodiments, the battery is a rechargeable battery, and the housing further includes an induction device for recharging the battery from a power supply externally of the subject's body.

A particular construction of a biosensor assembly is described for purposes of example, in which the biosensors are located in one side of the housing; the battery, antenna and induction device are located in the opposite side of the housing; and the reference-data storage device, the measured-data storage device, and the processor are carried by a printed circuit board located inbetween.

According to another aspect of the present invention, there is provided a health monitoring system comprising a biosensor assembly as described above; and a central station externally of the subject and including a receiver for receiving the data signals transmitted by the biosensor assembly and a central processor for processing the data signals. In the described preferred embodiment, there are a plurality of biosensor assemblies each implantable in the body of a different subject; and the central processor at the central station is designed to identify the biosensor assembly from which it is receiving data, and includes a central storage device for separately storing the data received from each biosensor assembly.

According to still further features in the described preferred embodiment, the central processor at the central station also includes a transmitter and receiver permitting two-way communication with respect to a plurality of caregiver terminals, enabling a caregiver at a respective terminal to receive the data measured from a particular subject and to transmit a response thereto for actuating an alarm, or for initiating a treatment, in response to the data received from the subject. The two-way communication with respect to the plurality of caregivers terminals may be via the cellular telephone network.

According to a still further feature in the described preferred embodiment, the system may further include an additional biosensor implantable in a different part of the subject's body and in communication in a wireless manner with the processor of the biosensor assembly implanted in the subject's body. Thus, the latter biosensor does not require a separate processor etc., but serves only to sense a medical parameter at the respective location of the subject's body and to communicate the sensed information to the biosensor assembly which includes the processor. This information is processed with the other information received, to determine whether an abnormal condition has arisen, and if so, to actuate an alarm, initiate a treatment, etc.

According to yet another aspect of the present invention, there is provided a method of monitoring a medical condition of a subject, or a plurality of subjects, utilizing the above-described biosensor assemblies.

As will be described more particularly below, the invention enables monitoring the health of subjects automatically and autonomously, i.e. without a conscious or active effort on the part of the subject. Moreover, the invention enables automatic and autonomic transfer and analysis of the measured parameters by medical personnel, and further, enables automatic and autonomic treatment should an emergency condition arise, for example, an electric shock treatment in case of a cardiac arrest.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is a schematical diagram illustrating one form of health monitoring system constructed in accordance with the present invention;

FIG. 2 is a schematical diagram more particularly illustrating the biosensor assembly in the system of FIG. 1;

FIG. 3 is a schematical diagram more particularly illustrating the central processor in the health monitoring system of FIG. 1;

FIG. 4 is a flowchart illustrating an example of the operation of the health monitoring system of FIGS. 1-3;

FIGS. 5-8 are pictorial illustrations, from various sides, of the biosensor assembly used in the health monitoring system of FIGS. 14;

FIG. 9 is an enlarged sectional view illustrating the construction of the biosensor assembly of FIGS. 5-8; and

FIG. 10 schematically illustrates a variation wherein the subject includes a plurality of biosensor assemblies implanted in various locations of the subject to sense various conditions.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF A PREFERRED EMBODIMENT

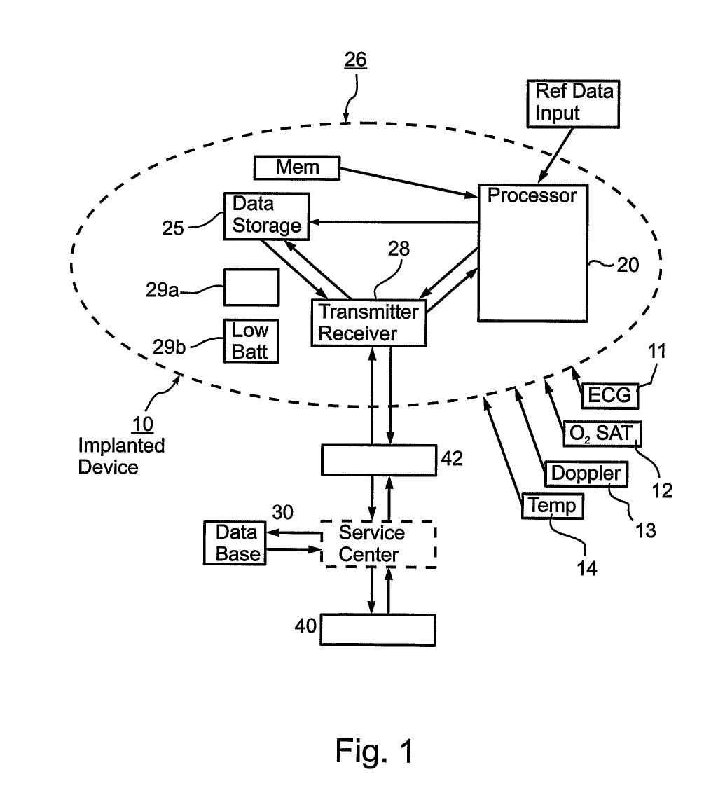

FIG. 1 schematically illustrates a preferred embodiment of the invention, which includes a biosensor assembly, schematically indicated within the broken-line block generally designated 10, having an implanted processor generally designated 20, communicating in a wireless manner with a central station proessor schematically indicated by broken-line block 30. It will be appreciated that the health monitoring system will include a plurality of biosensor assemblies 10 for implantation in many different subjects, all communicating with and served by the central station 30. Such communicating is a two-way communication, e.g. via the cellular telephone system and/or the GPS satellite system.

A plurality of terminals 40 by medical personal (e.g. physicians) or other medical caregivers, also have two-way communication with the central station processor 30, and via it, with the biosensor assemblies 10 implanted in the various individuals subscribing to this system. The two-way communication with the caregiver terminals 40 may also be via the cellular telephone system.

As further shown in FIG. 1, each biosensor assembly 10 includes a plurality of biosensors, indicated by blocks 11-14, providing inputs to processor 20 also implanted with the biosensors. In the example illustrated in FIG. 1, biosensor 11 is an ECG sensor for sensing and measuring the ECG signals of a subject; biosensor 12 is an oxygen saturation sensor for sensing and measuring the oxygen saturation of the subject's blood; sensor 13 is a Doppler sensor for sensing and measuring blood flow velocity in the subject; and biosensor 14 is a temperature sensor for sensing and measuring the temperature of the subject. Each sensor 11-14 may be of a known construction, and therefore further details of its construction or operation are not set forth herein.

Biosensors 11-14 measure their respective medical parameters in a continuous real-time manner and produce outputs, corresponding to the measured parameters, which outputs are continuously fed to processor 20 included within the implanted biosensor assembly 10.

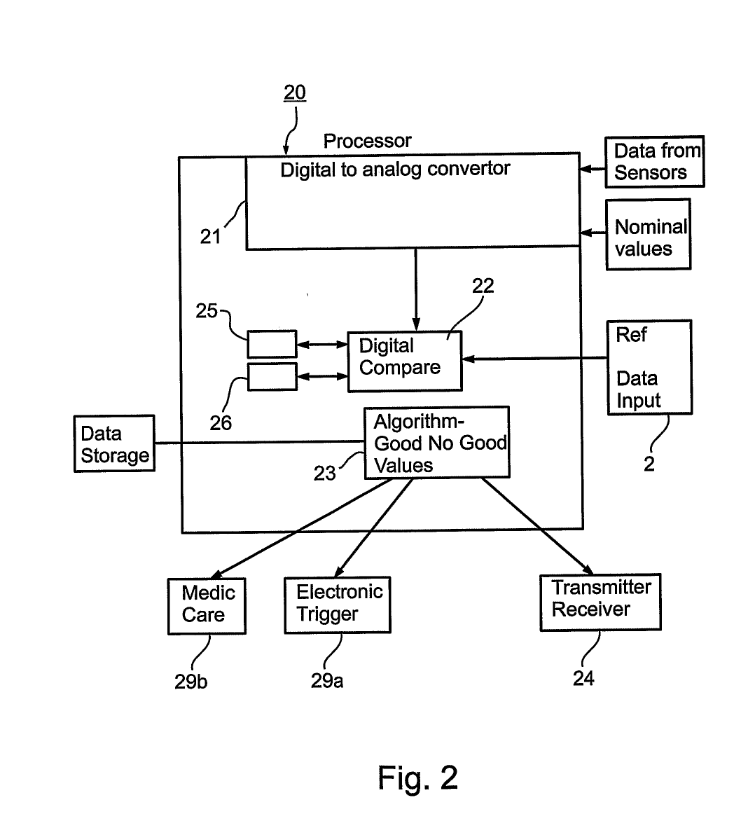

The implanted processor 20 continuously processes the information received from the biosensors 11-14, in a manner to be described more particularly below with respect to FIG. 2, and continuously stores data representing the measured medical parameters measured in a measured-data storage device 25. Implanted processor 20 also communicates with a reference-data storage device 26, which receives reference data, e.g. via an input 27, corresponding to a normal condition of the respective subject.

The measured-data processed by the implanted processor 20 is continuously compared with the reference data as stored in reference-data storage device 26 to determine whether the measured data sensed by biosensors 11-14, when compared to the reference data stored in the reference-data storage device 26, indicates a normal medical condition or an abnormal medical condition in the subject. If it is determined that the measured data indicates an abnormal medical condition, particularly an emergency condition, the implanted processor 20 actuates a transmitter 28 for transmitting a signal externally of the subject's body to indicate that an abnormal medical condition has been determined to be present.

As will be described more particularly below, the determination that an abnormal condition exists may be used for actuating transmitter 28 to transmit the measured data to the central station 30. Such an abnormal condition determination may also be used to actuate an alarm or a display to alert the subject that an abnormal condition has been found to exist, and to the subject to take remedial action; such a remedial action may be terminating or slowing-down in an exercise which the subject may then be doing. The signal outputted from the implanted processor 20, upon detecting an abnormal condition, may also be used to effect a treatment of the subject, e.g. to initiate an electric shock if a cardiac arrest has been determined to be present.

The abnormal condition, when found to be present, may also be displayed in the central station 30, and/or transmitted via the central station to the respective caregiver at one of the terminals 40, so that any action, deemed necessary or appropriate at the central station or at the caregiver terminal, can be immediately taken.

As shown in FIG. 1, the biosensor assembly 20 further includes a detector 27 for detecting when the measured-data storage device 25 is full, and another detector 28 for detecting a low battery condition. Either of the above conditions detected by detectors 27 and 28 will activate transmitter 24 to transmit the contents of the measured-data storage device 25 to the central station 30 so that such data will not be lost.

FIG. 1 also illustrates the optional inclusion of a relay unit, generally designated 42, which may be located externally of the subject's body, e.g. clipped to the subject's belt. Relay unit 42 includes a receiver for receiving the transmissions from transmitter 24, an amplifier for amplifying such transmissions, and another transmitter for re-transmitting this information to the central station 30.

FIG. 2 schematically illustrates the processor 20 carried by the implantable biosensor assembly 10 and implanted with it in the subject's body. As shown in FIG. 2, processor 20 includes a digital-to-analog converter 21 for converting to digital form the analog signals received from the biosensors 11-14. The so-measured parameters are stored in the measured-data storage device 25, and at the same time are fed to a comparator circuit 22 where the data is compared with the reference data stored in the reference-data storage device 26.

This comparison utilizes a diagnostic program, schematically illustrated by block 23, to determine whether the measured data, when compared to the reference data (serving as a base line representing a normal condition of the respective subject), indicates a normal medical condition or an abnormal medical condition in the subject. If a normal condition is indicated, processor 20 merely actuates transmitter 24 to transmit the measured data to the central station 30. However, if an abnormal condition is indicated, processor 20 transmits this data to the central station 30, but also actuates an alarm and/or display 29a at the central station to indicate the abnormal condition. Processor 20 may also initiate a treatment, as shown at 29b, in an attempt to remedy the abnormal condition, e.g. by applying an electrical shock in case a cardiac arrest is indicated as being present.

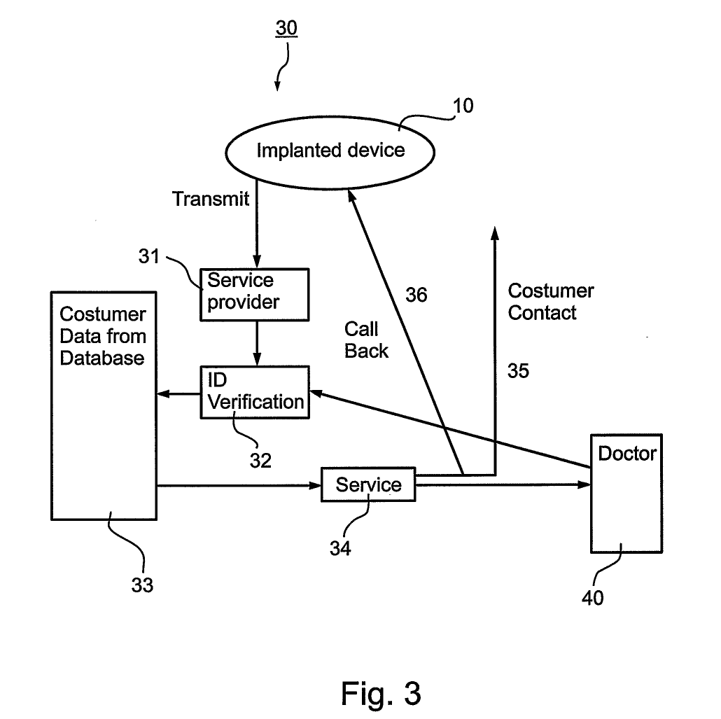

FIG. 3 illustrates the processor 30 at the central station, which communicates with the implanted biosensor assemblies 10 for each subject subscribing to the system. As indicated earlier, the communication is wireless, e.g. via the cellular telephone system, the GPS satellite system, etc.

Central station processor 30 includes a receiver 31 for receiving the data transmitted by the respective biosensor assembly 10, identifies the source of the data via an identification circuit 32, and stores the respective data in the storage device 33 according to the identification of the respective biosensor assembly.

Processor 30 in the central station further includes an interface, schematically indicated at 34, for the various terminals 40 of the caregivers (e.g. physicians), enabling each caregiver to communicate, e.g. by cellular telephone, with the processor. Thus, as schematically shown in FIG. 3, each caregiver terminal 40 may communicate with the database 33 via interface 34 to extract therefrom data for a particular-identified subject. Each caregiver terminal 40 may also communicate with each identified subject, via interface 34 and output line 35, to alert the subject as to a particularly dangerous condition, if detected, to provide instructions, etc.

Interface 34 may also be used to enable the subject himself (or herself) to communicate with the central station via the cellular telephone in order to extract data from its database 33 applicable to the respective subject. Such communication, e.g. by cellular telephone, can be effected via interface 34 and line 36, which interface and line can also be used for communicating an alarm to the subject immediately upon detecting an alarm condition,

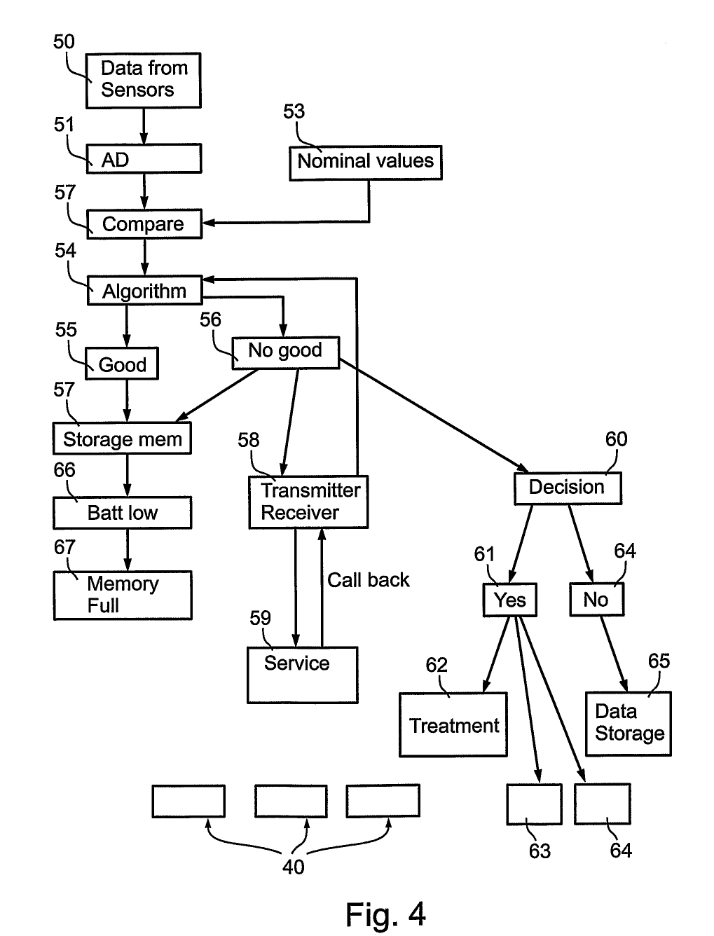

FIG. 4 is a flowchart illustrating the overall operation of the illustrated medical condition monitoring system. Thus, as described above, data is obtained in a continuous real-time manner from the biosensors 11-14 of each implanted biosensor assembly 10 (block 50), and is converted from analog form to digital form (block 51). This data is fed to a comparator (block 52), together with the normal reference values stored in the reference-data storage device 26 (block 53). Processor 20 then operates according to a known algorithm (block 54) to determine whether the measured data indicates a normal medical condition (block 55) or an abnormal medical condition (block 56). If a normal medical condition is indicated, the measured data is merely stored in the section of the measured-data storage device 25 allocated for the respective subject.

On the other hand, if an abnormal condition is indicated by block 56, transmitter 24 is activated (block 58) to transmit this data to the central station 30 (block 59). In addition, the processor makes a decision (block 60) as to whether some special action is required in view of the abnormal situation (block 61). The special action required may be the initiation of a treatment operation (block 62), and/or activating an alarm or display (block 63) to alert the subject to the abnormal condition. In either event, this abnormal data is also stored (block 64) so as to be entered into the medical history of the subject.

If no special action is required even though the measured data is abnormal (block 65), the data is merely stored so as to become part of the medical history of the subject.

Thus, as shown in the flowchart of FIG. 4, the transmitter of the respective biosensor assembly is activated to transmit the data to the central station whenever the measured data indicates an abnormal condition in the subject. As shown in FIG. 4, the transmitter is also activated whenever the measured-data storage device becomes full, or whenever the battery is determined to be low (block 68), so that no data will be lost under either of the above conditions.

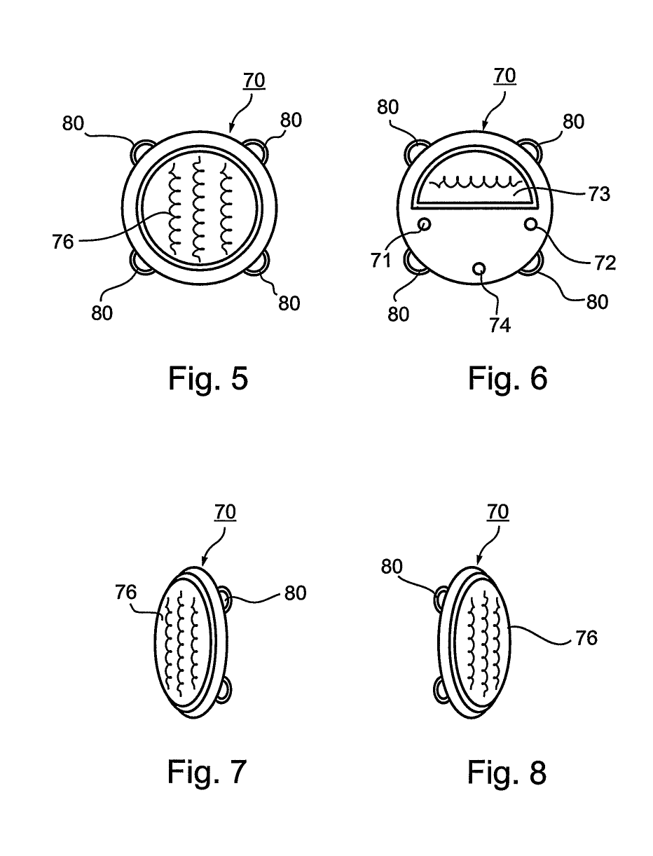

FIGS. 5-9 illustrate an example of a construction of an implantable biosensor assembly 20. The biosensor assembly therein illustrated includes a housing 70 sized and configured for implantation subcutaneously in the subject's body. Four biosensors, corresponding to biosensors 11-14 in FIG. 1, are carried on one side of the housing, namely that side illustrated in FIG. 6. These sensors include: an ECG sensor 71 for sensing and measuring the ECG signals of the subject; an oxygen-saturation sensor 72 for sensing and measuring the oxygen saturation of the subject's blood; a Doppler sensor 73 for sensing and measuring blood flow velocity in the subject; and a temperature sensor 74 for sensing and measuring the temperature of the subject. As indicated earlier known constructions may be used.

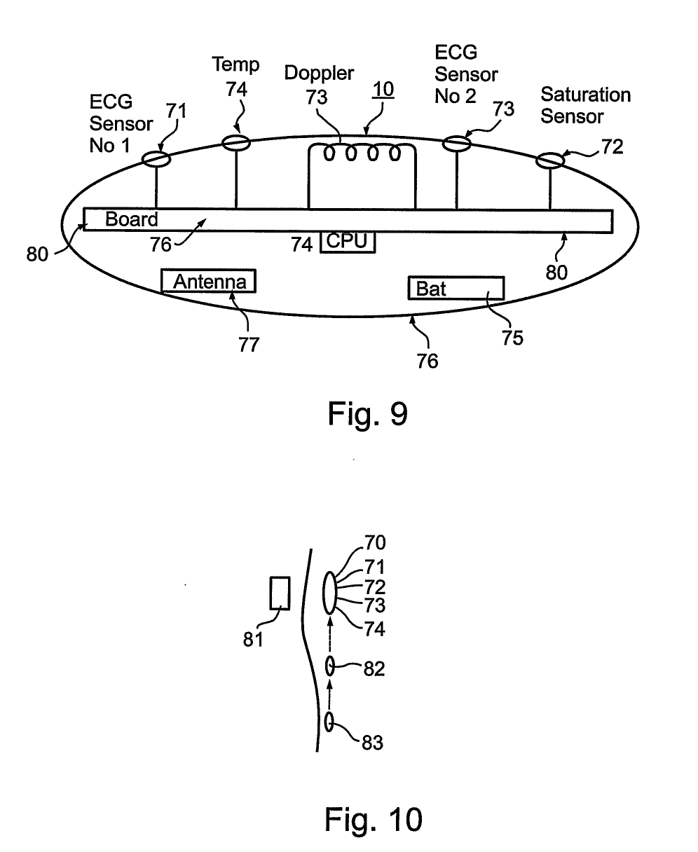

As shown particularly in FIG. 9, the opposite side of housing 70 contains a battery 75, an induction coil 76 for charging the battery, and an antenna 77 for transmitting the sensed and measured data to the central station 30. Housing 70 further contains a printed circuit board 78 mounting a CPU 79 which includes the transmitter (24, FIG. 1), the two storage devices 25, 26, and the remainder of the electrical circuitry as illustrated in FIG. 1.

As seen particularly in FIGS. 5 and 6, the biosensor assembly housing 70 is of circular configuration. It includes four outwardly-projecting loops 80 for receiving sutures in order to fix the assembly subcutaneously within the subject's body, when so fixed, the sensors 71-74 are exposed for contact with the appropriate tissues of the body, as shown in FIG. 10, while the induction coil 76 faces outwardly so as to enable its battery to be recharged by a battery charger 81 located externally of the body.

FIG. 10 also illustrates the possibility of implanting additional biosensors 82, 83, at different locations within the body. Each additional biosensor need not include a processor, since it can communicate its measured data with the main biosensor assembly, represented by housing 70, in a wireless manner as schematically shown in FIG. 10.

While the invention has been described with respect to a preferred embodiment, it will be appreciated that this is set forth merely for purposes of example, and that many other variations, modifications and applications of the invention may be made.

1. A biosensor assembly implantable in a subject for monitoring a medical condition of the subject, comprising:
at least one biosensor for measuring a medical parameter related to the medical condition;
a measured-data storage device for storing a measured data corresponding to the measured parameter;
a reference-data storage device for storing a reference data corresponding to a normal medical condition of the subject;
a transmitter including an antenna for transmitting a signal containing data externally of the subject;
a battery for powering said transmitter;
a processor for comparing the measured data with the reference data to identify an abnormal medical condition; and
a housing containing the at least one biosensor, the reference-data and measured-data storage devices, the transmitter, the battery, and the processor.

2. The biosensor assembly according to claim 1, including a plurality of biosensors for measuring a plurality of medical parameters and obtaining a plurality of medical data corresponding to the plurality of medical paramaters related to the medical condition.

3. The biosensor assembly according to claim 2, wherein said plurality of biosensors include an ECG sensor.

4. The biosensor assembly according to claim 2, wherein said plurality of biosensors include an oxygen-saturation sensor.

5. The biosensor assembly according to claim 2, wherein said plurality of biosensors include a Doppler sensor for measuring blood flow velocity.

6. The biosensor assembly according to claim 2, wherein said plurality of biosensors include a temperature sensor for sensing the temperature of the subject.

7. The biosensor assembly according to claim 2, wherein said processor utilizes the plurality of medical data to calculate a heart rate of the subject.

8. The biosensor assembly according to claim 2, wherein said processor utilizes the plurality of medical data to calculate a blood pressure of the subject.

9. The biosensor assembly according to claim 1, wherein said transmitter is a cellular modem for transmitting the signal via a cellular telephone network.

10. The biosensor assembly according to claim 9, further including a cellular modem receiver for receiving a signal via the cellular telephone network.

11. The biosensor assembly according to claim 1, wherein said transmitter is a GPS transmitter for transmitting the signal via the GPS satellite system.

12. The biosensor assembly according to claim 11, wherein said GPS transmitter signal includes data for identifying the biosensor assembly and its location.

13. The biosensor assembly according to claim 1, wherein said battery is a rechargeable battery and said housing further includes an induction device for recharging said battery from an external power supply.

14. The biosensor assembly according to claim 13, wherein said at least one biosensor is located on one side of the housing, said battery, antenna and induction device are located on an opposite side of said housing, and said reference-data storage device, measured-data storage device and processor are supported by a printed circuit board located between the one side and the opposite side.

15. The biosensor assembly according to claim 1, wherein said housing includes a loop for fixing said housing subcutaneously to the subject.

16. A health monitoring system, comprising:
a biosensor assembly for monitoring a medical condition of a subject including:
at least one biosensor for measuring a medical parameter related to the medical condition,
a measured-data storage device for storing a measured data corresponding to the measured parameter,
a reference-data storage device for storing a reference data corresponding to a normal medical condition of the subject,
a transmitter including an antenna for transmitting a signal containing data externally of the subject,
a processor for comparing the measured data with the reference data to identify an abnormal medical condition and to transmit in the signal an indication that the abnormal medical condition has been identified, and
a housing containing the at least one biosensor, the reference-data and measured-data storage devices, the transmitter, and the processor; and
a central station externally of the subject for processing the data contained in the signal transmitted by the biosensor assembly.

17. The health monitoring system according to claim 16, wherein said system includes a plurality of said biosensor assemblies for measuring medical conditions corresponding to each of a plurality of subjects, and said central station identifies each of the plurality of biosensor assemblies from which it receives a signal.

18. The health monitoring system according to claim 17, further including a terminal in electronic communication with said central station said terminal being configured to receive the data contained in the signal transmitted from one of the plurality of biosensor assemblies.

19. The health monitoring system according to claim 18, wherein said electronic communication is via a cellular telephone network.

20. The health monitoring system according to claim 16, wherein said system further includes a relay unit for receiving the signal transmitted by the biosensor assembly and transmitting an amplified signal to said central station corresponding to the signal transmitted by the biosensor assembly.

21. The health monitoring system according to claim 16, further including an additional biosensor for measuring a medical parameter, the additional biosensor being in wireless electronic communication with said biosensor assembly.

22. A method of monitoring a medical condition of a subject, comprising:
implanting in the subject a biosensor assembly including
at least one biosensor for measuring a medical parameter related to the medical condition,
a measured-data storage device for storing a measured data corresponding to the measured parameter,
a reference-data storage device for storing a reference data corresponding to a normal medical condition of the subject,
a transmitter including an antenna for transmitting a signal containing data externally of the subject,
a processor for processing data, and
a housing containing the at least one biosensor, the reference-data and measured-data storage devices the transmitter, and the processor;
comparing the measured data with the reference data to identify an abnormal medical condition; and
transmitting a signal externally of the subject indicating that an abnormal medical condition has been identified.

23. The method according to claim 22, wherein said housing includes a plurality of biosensors for measuring a plurality of medical parameters related to the medical condition of the subject.

24. (canceled)

25. The method according claim 22, further including the steps of monitoring the utilization of the measured-data storage device and transmitting the stored measured data upon determining said storage device is substantially full.

26. The method according to claim 22, further including the steps of:
providing a plurality of said biosensor assemblies each implantable in the body of a different subject;
providing a central station externally of the subjects for receiving the signal from at least one of the plurality of biosensor assemblies and for processing the data contained in the signal; and
identifying the biosensor assembly from which the central station received the signal.

27. The method according to claim 26, further including the steps of:
providing a terminal in electronic communication with said central station; and transmitting the data contained in the signal received by the central station to the terminal.

28. The method according to claim 27, wherein said transmission from the central station to the terminal is via a cellular telephone network.

29. (canceled)

30. The method according to claim 22, further including the steps of:
providing an additional biosensor for measuring a second medical parameter; and
transmitting a wireless signal from the additional biosensor to the biosensor assembly containing measured data corresponding to the second medical parameter.

31. The biosensor assembly according to claim 1, wherein said data contained in the signal includes an indication of an abnormal medical condition.

32. The biosensor assembly according to claim 1, wherein said data contained in the signal includes measurement data.

33. The biosensor assembly according to claim 1, wherein said data contained in the signal includes an alarm.

34. The biosensor assembly according to claim 2, wherein said processor utilizes the plurality of corresponding medical data to identify the abnormal medical condition.

35. The biosensor assembly according to claim 2, wherein said housing is sized and configured for subcutaneous implantation in the subject.

36. The health monitoring system according to claim 18, wherein said terminal is further adapted to transmit a command to each of the plurality of biosensor assemblies.

37. The health monitoring system according to claim 36, wherein said command is to actuate an alarm.

38. The health monitoring system according to claim 36, wherein said command is to initiate a treatment.

39. The health monitoring system according to claim 36, wherein said command is to transmit data corresponding to the measured parameters.

40. The method according to claim 23, wherein said processor utilizes the plurality of measured parameters to identify an abnormal medical condition.
